(19) United States
(12) Patent Application Publication
(10) Pub. No.: US 2026/0258071 A1
(43) Pub. Date: Sep. 3, 2026

(54) UV MICRO LED ARRAY DEVICE AS A SYNTHETIC DNA PROBE MANUFACTURING DEVICE AND DISPOSABLE DNA DETECTION DEVICE

(71) Applicant: Applied Materials, Inc., Santa Clara, CA (US)

(72) Inventors: Christopher Dennis BENCHER, Santa Clara, CA (US); Joseph R. JOHNSON, Redwood City, CA (US); Mingwei ZHU, San Jose, CA (US); Nag B. PATIBANDLA, Santa Clara, CA (US)

(21) Appl. No.: 19/357,961

(22) Filed: Oct. 14, 2025

Related U.S. Application Data
(60) Provisional application No. 63/716,606, filed on Nov. 5, 2024.
(60) Provisional application No. 63/788,739, filed on Apr. 14, 2025.

(51) Int. Cl. C07H 21/04; B01J 19/00

(52) U.S. Cl. CPC C07H 21/04; B01J 19/0046

(57) ABSTRACT

Embodiments of the present invention generally relate to devices and methods related to deoxyribonucleic acid (DNA) synthesis. A device for synthesizing DNA is provided. The device includes a LED panel, the LED panel includes a backplane, the backplane having a backplane surface, the backplane surface having at least one contact pad disposed over the backplane surface, a plurality of LED, the plurality of LEDs disposed over the backplane surface, where each LED of the plurality of LEDs couples to a contact pad, pixel isolation (PI) structures, the PI structures disposed over the backplane surface and between the plurality of LEDs, the PI structures defining a plurality of wells, and a coating disposed over the LED panel, the coating operable to seal each well to hold a liquid.

### CROSS-REFERENCE TO RELATED APPLICATIONS

This application claims the benefit of U.S. Provisional Application No. 63/788,739, filed on Apr. 14, 2025, and U.S. Provisional Application No. 63/716,606, filed on Nov. 5, 2024, each of which is incorporated by reference herein in its entirety.

### SEQUENCE LISTING

The present application contains a Sequence Listing submitted electronically in XML format via EFS-Web and is hereby incorporated by reference in its entirety. The Sequence Listing file is entitled ‘44025043US03_Sequence_Listing’, was created on May 11, 2026, and has a size of 2900 bytes.

### BACKGROUND

### Field

Embodiments of the present invention generally relate to devices and methods related to deoxyribonucleic acid (DNA) synthesis. Specifically, embodiments disclosed herein provide devices and methods for synthesizing DNA that incorporates a light emitting diode (LED) panel.

### Description of the Related Art

A light emitting diode (LED) panel uses an array of LEDs, with individual LEDs providing the individually controllable pixel elements that are incorporated in a variety of devices. The devices include, but are not limited to implemented biology devices for DNA synthesis, DNA sequencing, protein prototyping, photocontrolled polymer synthesis, 3D printing devices, drug discovery devices, LED displays, and any devices include that LED pixels having LEDs described herein. The devices may include LED of different sizes or LEDs of different wavelengths dependent on the implementation.

Current DNA sequencing involves photolithography devices and methods. Currently, one method to synthetically manufacture DNA includes using one UV lamp projects light through various photolithographic masks to reach a plate of many wells that include DNA components. A different photolithographic mask is required for each step of manufacturing the DNA components. Following the manufacturing of the DNA, imaging sources and devices must be used to detect the DNA that was manufactured.

Although current DNA manufacturing and detection processes represent a technological leap in DNA technology, challenges remain regarding DNA manufacturing and detection. For example, current DNA manufacturing and detection processes and devices require numerous steps that require constant change of manufacturing parts (e.g., changing the masks used in a photolithography system). This leads to an expensive and time consuming process that requires precise handling at each step. Accordingly, there is a need in the art for improved devices and methods related to DNA manufacturing and detection.

### SUMMARY

Embodiments of the present invention generally relate to devices and methods related to deoxyribonucleic acid (DNA) oligonucleic (oligo) array manufacturing and DNA sample detection. Specifically, embodiments disclosed herein provide devices and methods for manufacturing DNA and a detection device associated with DNA sequence detection that incorporate a light emitting diode (LED) panel.

Embodiments of the present invention generally relate to devices and methods related to deoxyribonucleic acid (DNA) synthesis. Specifically, embodiments disclosed herein provide devices and methods for synthesizing DNA that incorporates a light emitting diode (LED) panel.

In one embodiment, a device for synthesizing deoxyribonucleic acid (DNA) is provided. The device includes a LED panel, the LED panel includes a backplane, the backplane having a backplane surface, the backplane surface having at least one contact pad disposed over the backplane surface, a plurality of LED, the plurality of LEDs disposed over the backplane surface, where each LED of the plurality of LEDs couples to a contact pad, pixel isolation (PI) structures, the PI structures disposed over the backplane surface and between the plurality of LEDs, the PI structures defining a plurality of wells, and a coating disposed over the LED panel, the coating operable to seal each well to hold a liquid.

In another embodiment, a device is disclosed. The device including a LED panel, the LED panel including a backplane, the backplane having a backplane surface, the backplane surface having at least one contact pad disposed over the backplane surface, a plurality of LEDs, the plurality of LEDs disposed over the backplane surface, wherein each LED of the plurality of LEDs couples to a contact pad, and pixel isolation (PI) structures, the PI structures disposed over the backplane surface and between the plurality of LEDs, the PI structures defining a plurality of wells, wherein each LED of the plurality of LEDs disposed in each well of the plurality of wells is operable to anchor deoxyribonucleic acid (DNA) oligo array over the LED for DNA synthesis.

In another embodiments, a device is disclosed. The device includes a LED panel, the LED panel including a backplane, the backplane having a backplane surface, the backplane surface having at least one contact pad disposed over the backplane surface, a plurality of LEDs, the plurality of LEDs disposed over the backplane surface, wherein each LED of the plurality of LEDs couples to a contact pad; and pixel isolation (PI) structures, the PI structures disposed over the backplane surface and between the plurality of LEDs, the PI structures defining a plurality of wells, a coating disposed over the LED panel, the coating operable to seal each well to hold a liquid, and deoxyribonucleic acid (DNA) directly synthesized on each LED of the plurality of LEDs or synthesized over the coating disposed over the LED panel.

In another embodiment, a method for fabricating a deoxyribonucleic acid (DNA) is provided. The method includes applying a starting medium over a LED panel, applying a plurality of linker molecules over the LED panel, the linker molecule attaching over a LED, applying a plurality of photosensitive molecules over the LED panel, the plurality of photosensitive molecules being sensitive to a UV light, at least one photosensitive molecule of the plurality of photosensitive molecules binding to the plurality of linker molecules, cleaving the at least one photosensitive molecule of the plurality of photosensitive molecules from at least one linker molecule of the plurality of linker molecule with the UV light, applying a nucleotide solution over the LED panel, a nucleotide from the nucleotide solution attaching to at least one linker molecule of the plurality of linker molecules, rinsing the nucleotide solution from the LED panel, and applying the plurality of photosensitive molecules over the LED panel, at least one photosensitive molecule of the plurality of photosensitive molecules binding to the nucleotide.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A is a cross-sectional view of a LED panel, according to certain embodiments.

FIG. 1B is a top-view of a LED panel, according to certain embodiments.

FIG. 1C is a cross-sectional view of a LED panel with a cover, according to certain embodiments.

FIG. 2A is a schematic view of a test device including a LED panel, according to certain embodiments.

FIG. 2B is a schematic view of a test device reader, according to certain embodiments.

FIG. 3 is a flow diagram of a method of manufacturing DNA oligonucleotide arrays on a LED panel, according to certain embodiments.

FIGS. 4A-4G are cross-sectional views of a LED panel during DNA oligonucleotide array manufacturing, according to certain embodiments.

FIG. 5 is a flow diagram of a method of applying a sample to a LED panel, according to certain embodiments.

FIG. 6A is a top-view of a LED panel during application of a sample to a LED panel, according to certain embodiments.

FIGS. 6B-6F are cross-sectional views of a LED panel during application of a sample to a LED panel, according to certain embodiments.

FIG. 7 is a flow diagram of a method of detecting a sample, according to certain embodiments.

FIGS. 8A-8C are a schematic view of a test device and a test device reader during detection of a sample, according to certain embodiments.

FIG. 9 is a flow diagram of a method of synthesizing deoxyribonucleic acid (DNA), according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present invention generally relate to devices and methods related to deoxyribonucleic acid (DNA) oligonucleic (oligo) arrays manufacturing and DNA sample detection. Specifically, embodiments disclosed herein provide devices and methods for manufacturing DNA and a detection device associated with DNA sequence detection that incorporates a light emitting diode (LED) panel. The LED panel includes a plurality of wells and each well includes at least one LED disposed in the well. In some embodiments, the LED is a micro-LED. Each LED is operable to be controlled individually (e.g., each LED is operable to be switched on or off individually). The LED is operable to provide a UV light to DNA components to manufacture DNA. For example, DNA oligo arrays are formed over the surface of the LED by applying a starting medium over the surface of the LEDs, adding at least one linker molecule to each well, adding at least one photosensitive molecule to each well, turning on designated LEDs in individual wells to cleave the photosensitive molecule from the linker molecule, and turning off each designated LED. In one or more embodiments, the LED includes a coating or a layer so that a DNA oligo array is formed on the coating or layer over the LED. Cleaving the photosensitive molecule exposes the linker molecule to DNA nucleotides. A nucleotide solution is disposed over the LED panel and the nucleotides in the nucleotide solution will pair to each exposed linker molecule. Photosensitive molecules are reapplied to the LED panel, and the photosensitive molecules bind to the nucleotides. The process continues by cleaving and reapplying photosensitive molecules and applying with varying nucleotide solution according to the desired DNA oglionucleotide (oligo) array sequence until the DNA oligo array is complete. The DNA oligo array may be a DNA probe. The LED panel provides a method and device for controlled growth of individual DNA oligo array without the use of photolithography tools such as masks.

In some embodiments, the DNA oligo arrays disposed in each well are used to probe (pair with) particular single strand DNA sequences, where the single strange DNA sequence is the target DNA. The single strand DNA sequences are paired to a labeling probe with a UV excitable fluorophore. The labeling probe includes a complementary sequence of DNA such that it will pair to the single strand of DNA (the target DNA). A fluorophore emits light at a certain wavelength when the UV light emits towards the fluorophore. A sample includes at least one single strand of target DNA sequence where a labeling probe including the UV excitable fluorophores is paired to the at least one single strand DNA sequence. The sample is disposed over the LED panel, which includes at least one DNA oligo array in each well. A single strand of DNA will pair to a DNA oligo array with a corresponding nucleotide, causing the single strand DNA to be bound within the respective well. The LED panel including DNA samples disposed into at least one well is disposed into a device (e.g., a test device). The device includes a photo-diode and a UV filter. The photo-diode and the UV filter are disposed over the LED panel. The LEDs disposed in each well are sequentially switched on to excite the fluorophore present in the well, if any. The fluorophores emit light at a particular wavelength once they are excited by UV light, and the light emitted by the fluorophores is a signal to the photo-diode. The photo-diode, disposed over the LED panel, receives the signal from the fluorophores and converts the signal to a result for a user. The result will display if a particular DNA sequence was detected within a particular well of the LED panel. This allows for easy, cost effective detection of particular DNA sequences while minimizing the equipment needed to produce a result.

### Device Embodiments

### LED Panel Device

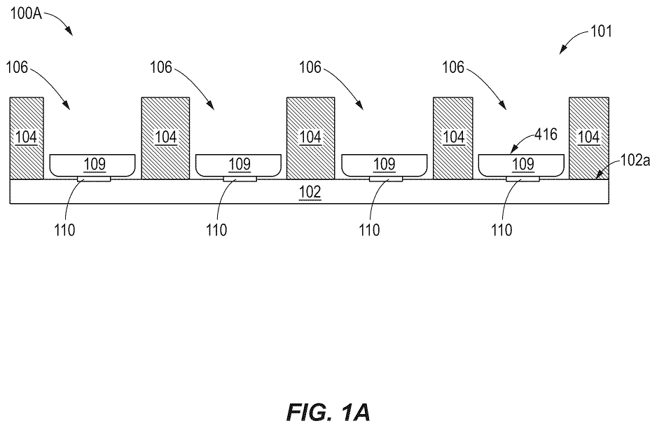

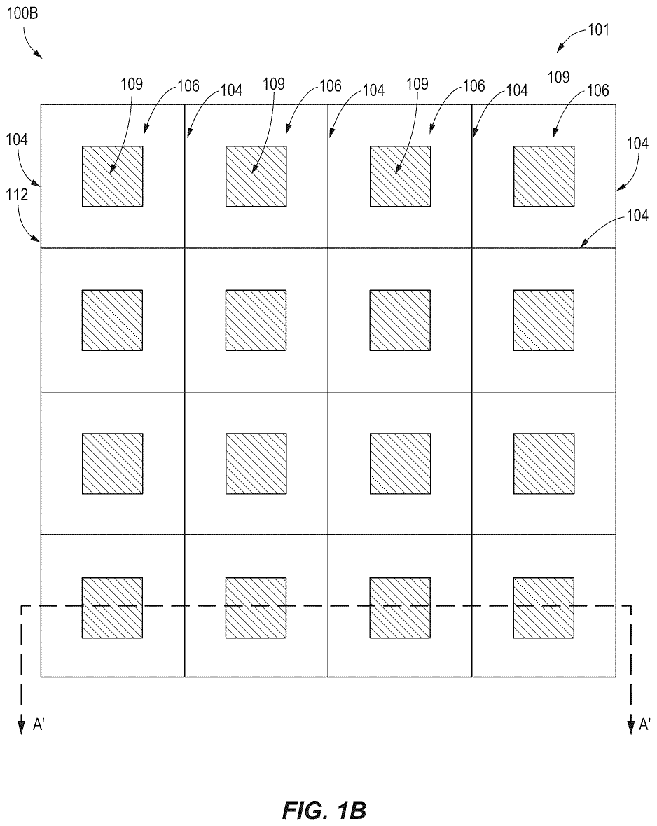

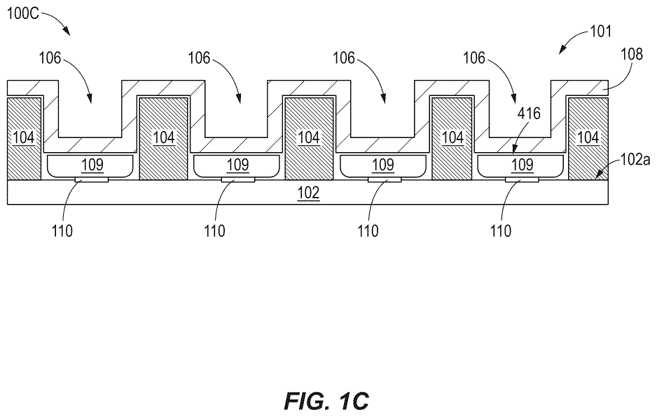

FIG. 1A is a cross-sectional view 100A of a LED panel 101. FIG. 1B is a top-view 100B of the LED panel 101. FIG. 1A shows a cross section along line A′ to A′ shown in FIG. 1B. FIG. 1C is a cross-sectional view 100C of a LED panel 101 with a cover 108. The backplane 102 includes a backplane surface 102a. The backplane surface 102a includes a plurality of contact pads 110. The backplane surface 102a includes a plurality of LEDs 109, each LED of the plurality of LEDs 109 is disposed over a contact pad 110. The LEDs 109 include pairs of backplane electrodes (not pictured) and LED electrodes (not pictured). The LEDs of the present disclosure may be micro-LEDs. In one or more embodiments, the LEDs 109 are flip-chip LEDs. The flip-chip LEDs includes both electrodes on the same side of the LED and contact the backplane 102. In one or more embodiments including micro-LEDs, the micro-LEDs may be vertical die micro-LEDs, where a first electrode contacts the backplane and a second electrode contacts a top of the die. In one or more embodiments including micro-LEDs, the micro-LEDs may be lateral die micro-LEDs, where both electrodes are disposed over the top of the die after it is bonded to the backplane 102.

The LEDs 109 are integrated with backplane circuitry so that each LED 109 can be individually addressed. For example, the circuitry of the backplane can include a TFT active matrix array with a thin-film transistor and a storage capacitor (not illustrated) for each LED 109, column address and row address lines, column and row drivers, to drive the LEDs 109. Alternatively, the LEDs 109 can be driven by a passive matrix in the backplane circuitry. The backplane 102 can be fabricated using conventional complementary metal-oxide silicon (CMOS) process. Each LED 109 is configured to emit low wavelength light in a wavelength range. For example, the low wavelength light emits in a wavelength range of about 365 nm to about 450 nm. In one or more embodiments, the each LED 109 is configured to emit UV light. The size of the LEDs 109 may be about 30 um by 30 μm to about 100 um by 100 μm. In one or more embodiments, the surface 416 of each LED 109 is glass. In one or more embodiments, a transparent glass surface (not pictured) may be disposed over the LEDs 109.

Pixel isolation (PI) structures 104 are disposed over the backplane surface 102a. The PI structures 104 are disposed around the LEDs 109. The PI structures 104 may include organic material. The organic material may be an epoxy-based photoresist or a negative tone photoresist. The PI structures 104 are operable to filter the UV light 408 (e.g., the PI structures 104 limits or blocks cross talk of UV light between wells 106). In one or more embodiments, the PI structures include a black and/or opaque resist material. In one or more embodiments, the PI structures are operable to limit or block the crosstalk between wells 106 including excited fluorophores (a first fluorophore 606 and a second fluorophore 608). Adjacent PI structures 104 form a plurality of wells 106 around the LEDs 109. For example, one LED 109 is disposed into each well 106. In one or more embodiments, more than one LED is disposed into each well 106. In one or more embodiments including an intermediate layer is disposed over the LEDs 109, the intermediate layer includes but is not limited to glass or SiO₂. In embodiments including the intermediate layer, the PI structures are disposed over the transparent glass surface. A liquid proof material (not pictured) is disposed over the LED panel 101. The liquid proof material seals the circuitry of the LED panel 101 (e.g., the back plane circuitry) to provide protection from liquid material during any of the methods disclosed herein. Further, the liquid proof material allows for a well 106 to be leak proof (e.g., samples disposed in each well 106 will not transfer to adjacent wells). In one or more embodiments, a SiO₂ containing film may be disposed over each LED 109 within each well of the plurality of wells 106. The SiO₂ film provides a starting material for the DNA oligos to form on (e.g., connect to or contact during the beginning of the synthesis process as seen in Method 300). Further, the SiO₂ film provides a protective film to prevent liquid from interfering with the backplane circuitry connected to each LED 109.

As shown in FIG. 1A, one LED 109 is disposed at the bottom of each well 106 over the backplane surface 102a. Each well 106 includes a void space above the LED 109 defined by the PI structures 104. As shown in FIG. 1B, each well 106 includes a similar size. The PI structures 104 provide a repeating rectangular pattern around each LED 109. Further, the PI structures 104 define the outer wall 112 of the LED panel 101. As shown in FIG. 1B, the LED panel 101 includes sixteen wells 106. However, it is contemplated each LED panel 101 can include at least hundreds of wells 106. Each LED 109 is shown as a rectangular shape. However, it is contemplated that the LEDs 109 may be any shape.

In one or more embodiments, additional components may be disposed into each well 106 of the LED panel 101 with the LED 109 or in place of the LED 109. For example, micro-optical or micro-mechanical devices may be disposed within the well 106. For example, a photo-diode 210 is disposed within each well 106, the photo-diode 210 coated with a UV filter (e.g., UV filter 208). For example, a thermistor is disposed within each well 106 to provide thermal heating. In one or more embodiments, a piezo-electric vibrator operable to mix solutions or samples (e.g., a nucleotide solution 410 or a sample 610) during the methods disclosed below (e.g., method 300 or method 500) is coupled to the LED panel 101. In one or more embodiments, a piezo-electric vibrator operable to assist in distribution of the sample 610, nucleotide solution 410 or rinsing fluid during method 300 or method 500 is coupled to the LED panel 101. In one or more embodiments, a set of electrodes operable to apply a positive or negative electrical bias to each well 106 allows for pixel specific electro-chemical reactions.

In one or more embodiments, as shown in FIG. 1C, the LED panel 101 further includes a cover 108. The cover 108 is operable to fit over and around each well 106 and above each LED 109, as shown in FIG. 1C. The cover 108 is operable to be removable from the LED panel 101. The cover 108 provides a liquid proof protection from chemistry in each well 106. The chemistry occurs within the cover 108 and over each LED 109. For example, DNA synthesis or testing occurs over the cover 108 that is disposed within each well 106. The cover 108 may be removed after every use in order to provide an uncontaminated surface within each well 106 to perform any of the methods of the present disclosure (e.g., method 300 or method 500). In one or more embodiments, the cover 108 is a liner. In one or more embodiments, the cover 108 includes a transparent material operable to transmit UV light. In one or more embodiments, the cover 108 is a rigid material. For example, the cover 108 includes glass, sapphire, or combinations thereof. The cover 108 is etched into a shape that fits over the LED panel 101 (e.g., into each well 106). In one or more embodiments, the cover 108 further includes isolation structures between each well 106. The isolation structures are operably to prevent crosstalk through the cover 108 and between each well 106.

### Test Device

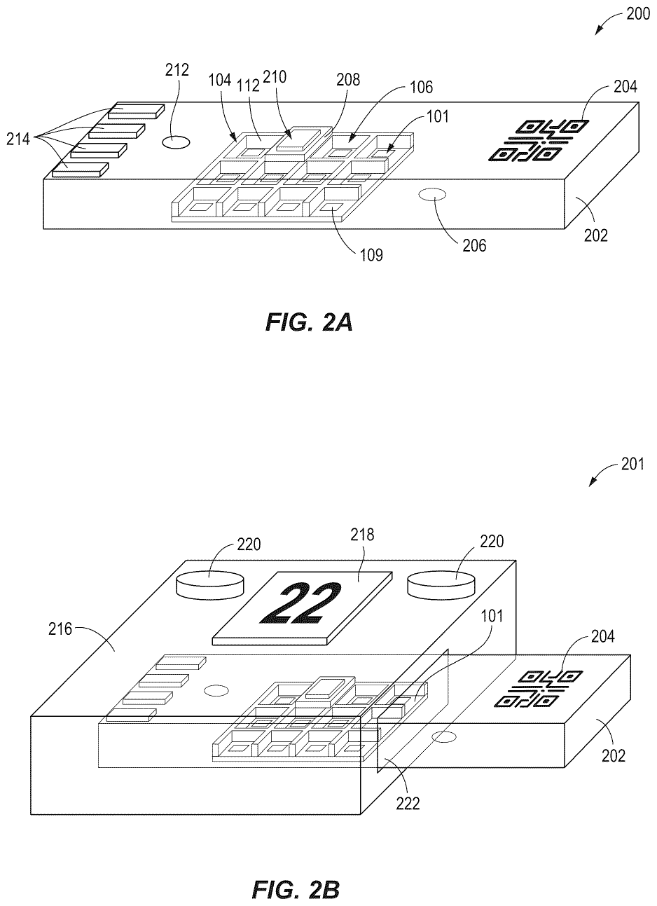

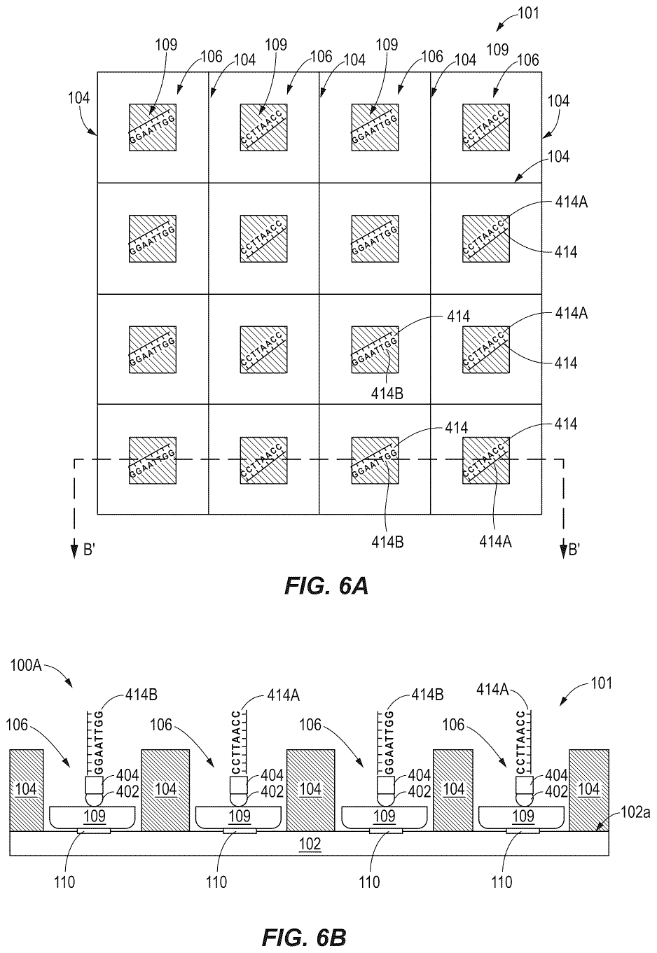

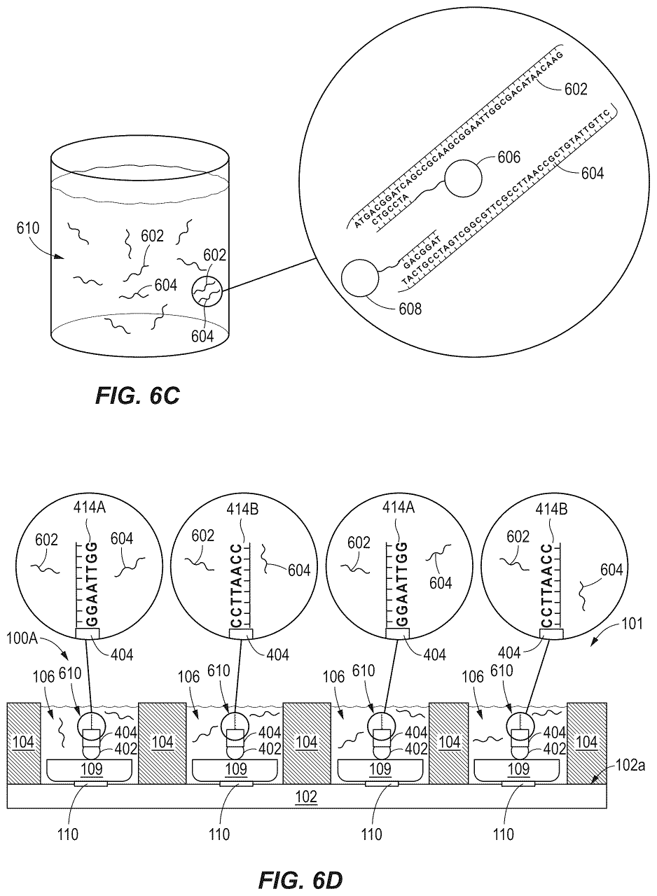

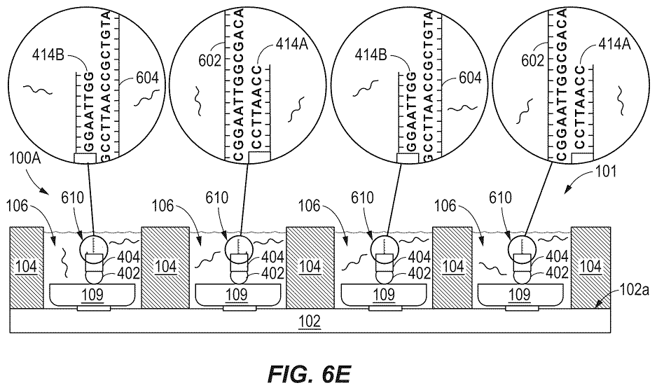

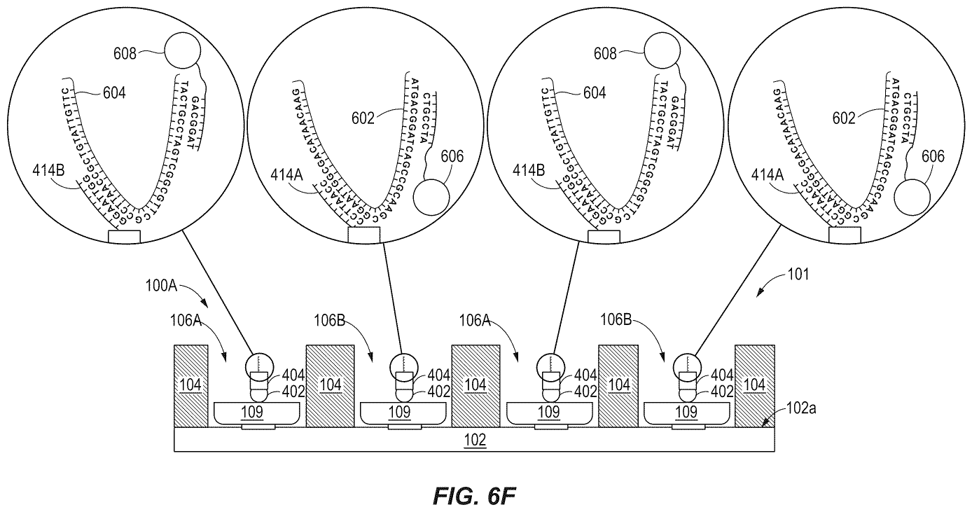

FIG. 2A is a schematic view of a test device 200 including a LED panel 101. Each test device 200 includes an identifying code 204, a drain orifice 206, the LED panel 101, a UV filter 208, a photosensitive device such as a photo-diode 210, a dispensing orifice 212, and a plurality of contact pins 214. The identifying code 204 allows for a user to identify information about the test device 200. For example, the user can identify what type of bacteria can be identified by the test device 200 or the name of the test device 200. The drain orifice 206 provides an opening for a sample 610 to exit the test device 200. The sample 610 (shown in FIG. 6C) may exit the test device 200 due to overflow after each well 106 is filled with sample 610 or during removal of the sample 610. The LED panel 101 is described above. The LED panel 101 is disposed within the test device 200. For example, the LED panel 101 is enclosed by the outside structure 202 of the test device 200. As shown in FIG. 2A, the LED panel 101 is shown with a cut away of the PI structure 104 to show the well 106. The LED panel 101 includes an outer wall 112 that encloses the LED panel 101. The UV filter 208 is disposed over the LED panel 101. The UV filter 208 protects the photo-diode 210 from UV light emitted from the LEDs 109 during use of the test device 200. The photo-diode 210 is disposed over the UV filter 208. The photo-diode 210 is operable to detect light emitted from a fluorophore (e.g., a first fluorophore 606 or a second fluorophore 608) that is attached to a DNA sample (e.g., the sample 610). The dispensing orifice 212 provides an opening to administer the sample into the test device 200 and the LED panel 101. The plurality of contact pins 214 are operable to connect the test device 200 to a test device reader 216. In one or more embodiments, the test device 200 is disposable. In one or more embodiments, the plurality of contact pins 214 are a universal serial bus (USB) or micro-USB connector. In the one or more embodiments with a USB or micro-USB, the test device 200 is operable to connect to a computer or smart phone.

FIG. 2B is a schematic view of a test device reader 216. As shown in FIG. 2B, the test device 200 as described above is inserted into the test device reader 216 to form a DNA sampling system 201. The test device reader 216 includes a switch 220, a test device opening 222 and a display 218. In one or more embodiments the test device reader 216 includes one switch 220 to turn the test device reader 216 on or off. In one or more embodiments a plurality of switches 220 are disposed on the test device reader 216. The test device opening 222 provides an opening to insert the test device 200 into the test device reader 216. The plurality of contact pins 214 disposed on the test device 200 connect to the test device reader 216. The connection between the contact pins 214 and the test device reader 216 is an electrical connection. The electrical connection provides electricity to the DNA sampling system 201. The electricity is used to power the LEDs and the photo-diode 210. The display 218 displays the results captured by the DNA sampling system 201 to the user.

### Methods

### Method of Manufacturing DNA Oligonucleotide (Oligo) Arrays

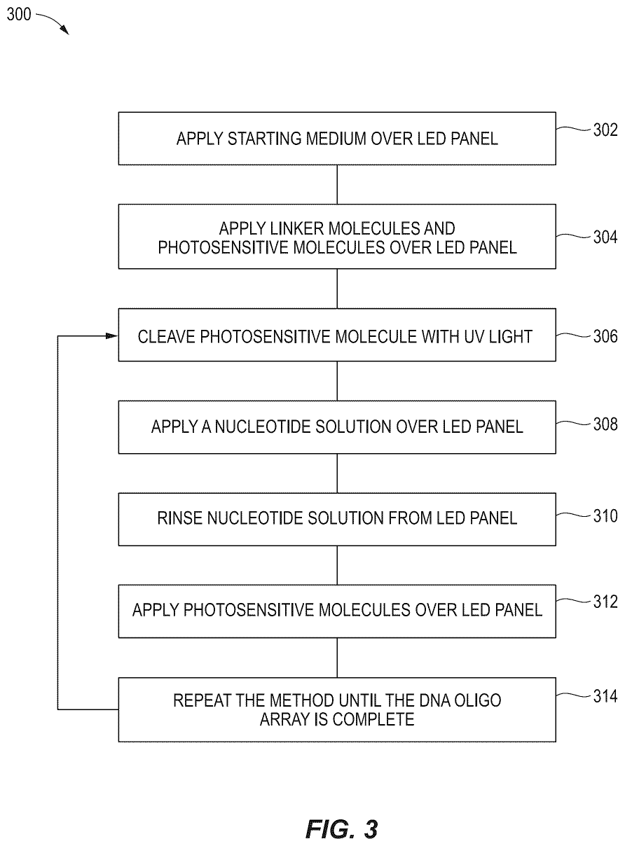

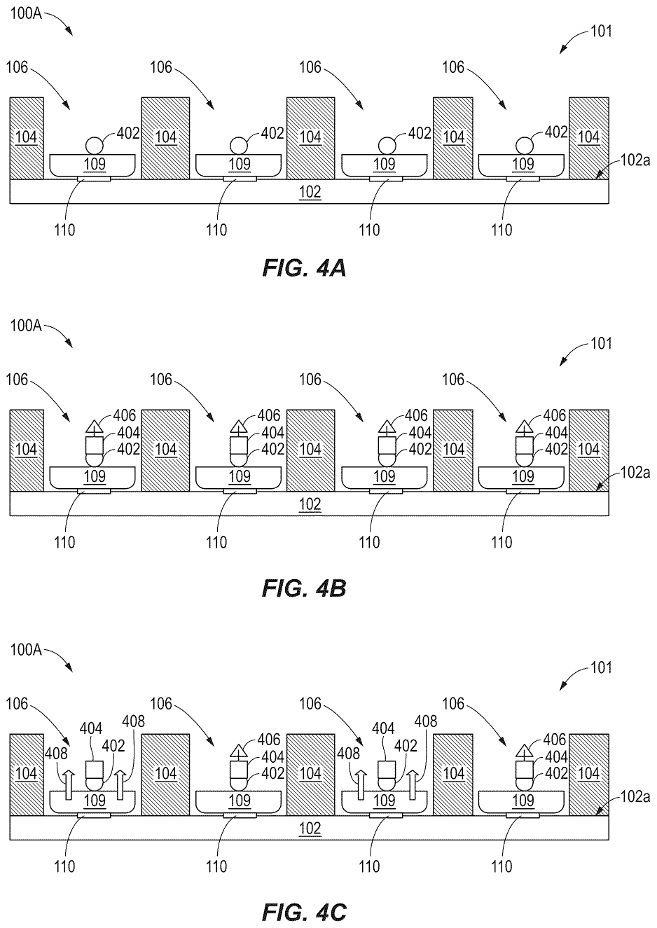

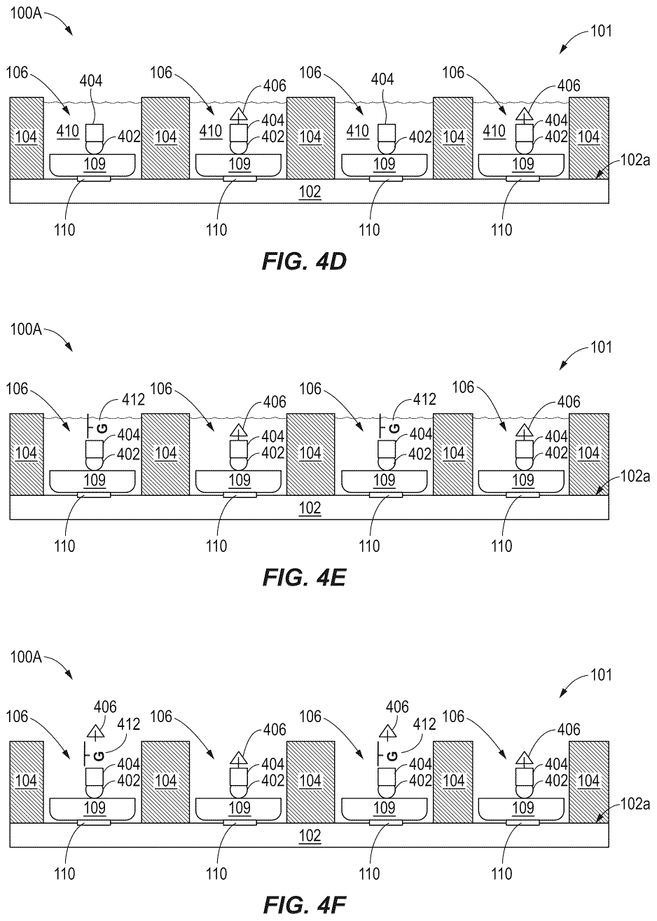

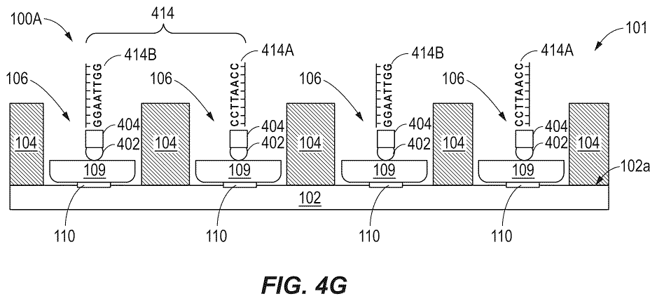

FIG. 3 is a flow diagram of a method 300 of manufacturing DNA oligonucleotide (oligo) arrays on a LED panel 101. The method 300 includes growing a DNA oligo array 414 by sequentially adding one nucleotide 412 at a time to the DNA oligo array 414 (e.g., DNA nucleotides adenine (A), thymine (T), guanine (G), and cytosine (C) or RNA nucleotides adenine (A), uracil (U), guanine (G), and cytosine (C)). FIGS. 4A-4G are cross-sectional views of a LED panel 101 during method 300. The DNA oligo array 414 is grown according to a predetermined sequence. As shown in FIG. 4G, a first DNA oligo array 414A and a second DNA oligo array 414B are used as examples of sequences. However, it should be understood that the predetermined sequence may be any DNA oligo array 414 sequence and may include any desired number of nucleotides 412.

At operation 302, as shown in FIG. 4A, a starting medium 402 is applied to the LED panel 101. The starting medium 402 is disposed into each well 106 over each LED 109. The starting medium 402 may include beads with DNA oligo array 414 starting sequences attached. Additionally or alternatively, the starting medium 402 may include silane to silate the surface of the LEDs 109 or the backplane surface 102a. Silating the surface includes applying a silane solution into each well 106 of the LED panel 101. Applying silane solution to each well 106 may include submerging the LED panel 101 into a silane solution, but any reasonable means of disposing a silane solution into each well 106 may be used. The silane will bond to the surface 416 of the LED 109. The LED 109 may include, but is not limited to, a III-V surface such as gallium-nitride. In one or more embodiments, each LED 109 includes a liquid barrier film. The liquid barrier film may be a glass film. In embodiments with a liquid barrier film, the silane will bond the liquid barrier film. In one or more embodiments, a SiO₂ containing film may be disposed over each LED 109 within each well of the plurality of wells 106. The SiO₂ film provides a starting material for the DNA oligos to form on. The SiO₂ containing film is deposited onto the LED panel 101 via chemical vapor deposition (CVD) or atomic layer deposition (ALD). In one or more embodiments, the surface of each LED is coated in a protein with a high affinity for biotin. In an embodiment including a protein with a high affinity for biotin, a biotin molecule is attached to the DNA to allow for the DNA to attach to the LED coated in the protein with a high affinity for biotin. In one or more embodiments, a gold and/or indium bond pad secures the LEDs 109 to the backplane 102. In an embodiment including a gold and/or indium bond pad, a thiol group is attached to the DNA and the thiol group attaches to the gold and/or indium bond pad such that the DNA is connected over the surface of the LED 109. In one or more embodiments, a cross-linker such as EDC (1-Ethyl-3-(3-dimethylaminopropyl) Carbodiimide Hydrochloride) is used to create a bond between phosphate groups associate with DNA and an amine. In embodiments using a cross-linker, the surface of each LED 109 is coated with an amine coated molecule so that DNA is connected over the surface of the LED 109.

At operation 304, a linker molecule 404 and a photosensitive molecule 406 is applied to the starting medium 402 in each well 106 of the LED panel 101. Applying linker molecules 404 or photosensitive molecules 406 to each well 106 may include submerging the LED panel 101 into linker molecule 404 solution or a photosensitive molecule 406 solution, respectively, but any reasonable means of disposing the linker molecules 404 or photosensitive molecules 406 into each well 106 may be used. The linker molecule 404 provides a start point for nucleotides 412 to attach during DNA oligo array 414 formation. The photosensitive molecule 406 protects a DNA oligo array 414 from additional nucleotide additions during DNA oligo array 414 formation. As shown in FIG. 4B, the linker molecule 404 attaches to a starting medium 402. Initially, the photosensitive molecule 406 attaches to the linker molecule 404. The starting medium 402 and the linker molecule 404 provide the means for the DNA oligo array 414 to secure to the surface 416 of the LED 109.

At operation 306, the photosensitive molecule 406 is cleaved from the linker molecule 404 by UV light 408. Each LED 109 is controlled individually and emits the UV light 408 independently. The PI structures 104 prevent cross talk of UV light 408 between adjacent wells 106. Controlling the UV light 408 allows for the control of cleaving the photosensitive molecule 406 which allows for individualized control of DNA oligo array 414 growth in each well 106. For example, as shown in FIG. 4C, UV light 408 is emitted in two wells 106. The UV light 408 cleaves the photosensitive molecules 406 from the linker molecule 404 in each well 106 exposed to UV light 408. In the example shown in FIG. 4C, one DNA oligo array 414 is represented. However, it is to be understood that millions of DNA oligo array 414 may be grown in each well 106 during Method 300.

At operation 308, a nucleotide solution 410 is applied over the LED panel 101. The nucleotide solution 410 is disposed over at least the wells 106. The nucleotide solution 410 may be applied over the LED panel 101 by submerging the LED panel 101 into the nucleotide solution 410 but any reasonable means of disposing a nucleotide solution 410 into each well 106 may be used. The nucleotide solution 410 may include any nucleotide (e.g., adenine (A), thymine (T), guanine (G), and cytosine (C)). The type of nucleotide solution 410 is determined by the desired sequence of DNA oligo array 414. As shown in FIG. 4D, the nucleotide solution 410 is disposed into every well 106. Additionally or alternatively, the nucleotide solution 410 may be selectively applied to wells 106. As shown in FIG. 4E, a G nucleotide 412 has bonded to the linker molecule 404. In this example, the G nucleotide 412 is the first nucleotide in the second DNA oligo array 414B. Additionally or alternatively, any nucleotide 412 may be the first nucleotide 412 in a DNA oligo array (e.g., the first DNA oligo array 414A includes a C as the first nucleotide 412 of the sequence).

At operation 310, the nucleotide solution 410 is rinsed form the LED panel 101. As shown in FIG. 4F, the nucleotide solution 410 has been removed from each well 106 of the LED panel 101. The G nucleotide 412, discussed above, remains bonded to the linker molecule 404. The nucleotide solution 410 may be rinsed from the LED panel 101 by any reasonable means in order to remove any remaining nucleotides 412 from the LED panel 101. At operation 312, a photosensitive molecule 406 solution is deposited into each well 106 of the LED panel 101. The photosensitive molecule 406 bonds to the nucleotide 412. As shown in FIG. 4F, the photosensitive molecule 406 bonds to the G nucleotide 412 in the wells 106. In one or more embodiments, operation 308, 310, and 312 occur simultaneously. For example, the nucleotide solution 410 includes nucleotides 412 paired with the photosensitive molecule 406 so that one solution can be used to grow the DNA oligo array 414.

At operation 314, the method 300 is repeated until the desired DNA oligo array 414 is formed. For example, the LED 109 in a desired well is switched on to emit UV light 408 to cleave the photosensitive molecule 406 from the desired DNA oligo array 414. A nucleotide solution 410 is applied over the wells 106, and the nucleotide 412 binds to the exposed nucleotide 412. For example, a second G nucleotide 412 binds to the G nucleotide 412 attached to the linker molecule 404 as the next nucleotide 412 in the sequence to form the second DNA oligo array 414B. The method 300 repeats sequentially until the desired DNA oligo array 414 is formed (e.g., the first DNA oligo array 414A or the second DNA oligo array 414B). Once the final nucleotide 412 binds to the DNA oligo array 414, there are no further operations (e.g., no photosensitive molecule binds to the last nucleotide 412 of the sequence). As shown in FIG. 4G, the completed DNA oligo arrays 414 include 8 nucleotides as an example. However, it is contemplated that the DNA oligo arrays 414 can include any number of nucleotides in any sequence. The completed DNA oligo arrays 414 are used as DNA probes for DNA sample testing. For example, the LED panel 101 including the completed DNA oligo arrays 414 may be disposed within a test device 200 as shown in method 700. Additionally or alternatively, the completed DNA oligo arrays 414 grown in the LED panel 101 may be cleaved from the LEDs 109.

### Method of Synthesizing DNA

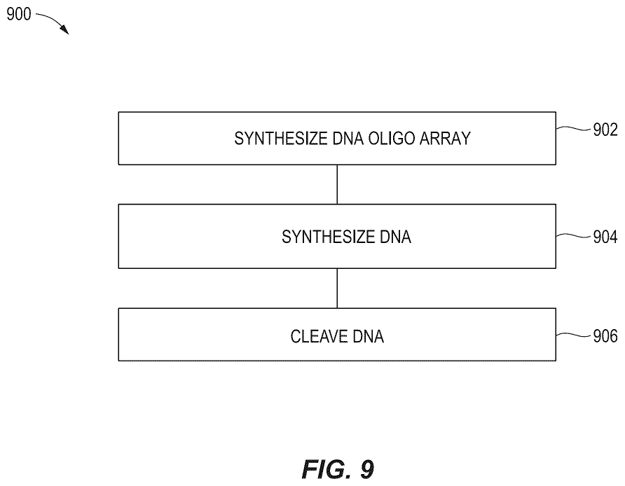

FIG. 9 is a flow diagram of a method 900 of synthesizing DNA on a LED panel 101. The method 900 includes synthesizing a DNA oligo array in each LED well. Each LED well may include the same DNA oligo array or different DNA oligo arrays according to the desired production requirements of the DNA synthesis.

At operation 902, the DNA oligo array is synthesized according to the above disclosed method 300. Method 300 continues until a complete DNA oligo array is synthesized (e.g., a complete DNA oligo array includes all the nucleotides required to form the desired DNA). Any combinations of nucleotides may be used to form the single strand of DNA. The DNA oligo array remains anchored, attached, secured, or bonded to the LED 109 or a coating over the LED 109.

At operation 904, the DNA is synthesized. A nucleotide solution 410 is disposed in each well 106 of the LED panel 101. The nucleotide solution 410 may be applied over the LED panel 101 by submerging the LED panel 101 into the nucleotide solution 410 but any reasonable means of disposing a nucleotide solution 410 into each well 106 may be used. The nucleotide solution 410 may include one type of nucleotide or a mixture of multiple nucleotides. The nucleotides in the nucleotide solution 410 form phosphodiester bonds with the DNA oligo array anchored to the LED 109 in each well 106 according to their complementary nucleotide. The paired nucleotides form the DNA.

At operation 906, the DNA is cleaved from each well 106. In one or more embodiments, the DNA is washed from the LED panel 101.

### Method of Applying a Sample to a LED Panel

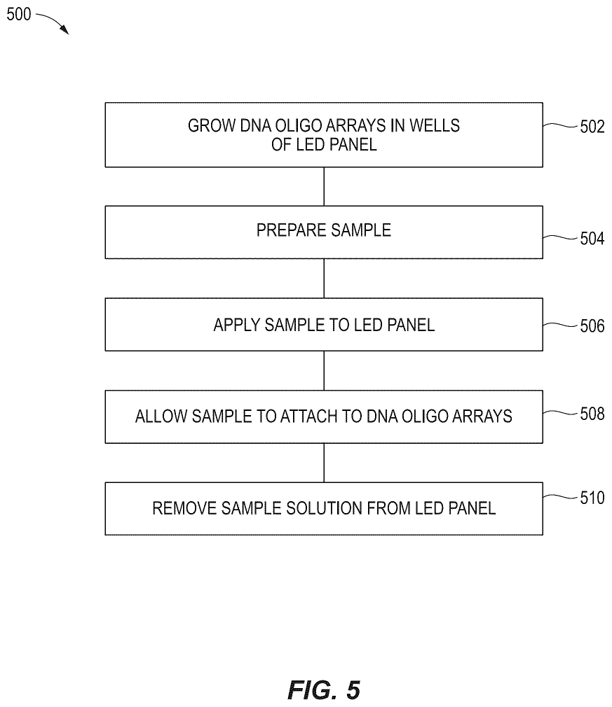

FIG. 5 is a flow diagram of a method 500 of applying a sample 610 to a LED panel 101. The method 500 includes preparing a sample 610 consisting of DNA (e.g., a first single strand of DNA 602 or a second single strand of DNA 604) for testing by applying the sample 610 to the LED panel 101. Additionally or alternatively, the sample 610 may include RNA. FIG. 6A is a top-view of the LED panel 101 during application of a sample 610 to the LED panel. 6B-6F are cross-sectional views of the LED panel 101 during application of a sample 610 to a LED panel 101. The sample 610 includes at least one single strand of DNA and at least one fluorophore. As shown in FIG. 6C, a first single stand DNA 602 and a second single strand of DNA 604 are shown in sample 610 and are used as example DNA samples. However, it should be understood that any DNA samples may be used. In one or more embodiments, as shown in FIG. 6C, the first single strand of DNA 602 includes a sequence of Seq ID No. 1. In one or more embodiments, as shown in FIG. 6C, the second single strand of DNA 604 includes a sequence of Seq. ID. No. 2.

At operation 502, DNA oligo arrays 414 are grown in the LED panel 101. FIG. 6A is a top-view 100B of the LED panel 101 including DNA oligo arrays 414. FIG. 6B shows a cross section alone line B′ to B′ shown in FIG. 6A including DNA oligo arrays 414. The DNA oligo arrays 414 are grown in the LED panel 101 according to method 300 described above. The DNA oligo arrays 414 are DNA probes specific to the desired DNA sequences in the sample 610. For example, the DNA oligo arrays 414 include a complementary sequence to a portion of the DNA sample (e.g., a first single strand of DNA 602 or a second single strand of DNA 604). As shown in FIG. 6A, the DNA oligo arrays 414 are disposed within each well 106 of the LED panel 101. The DNA oligo arrays 414 are enlarged to show the nucleotide sequence. As shown in FIGS. 6A and 6B a first DNA oligo array 414A and a second DNA oligo array 414B are disposed individually in a well 106. Both the first DNA oligo array 414A and the second DNA oligo array 414B are DNA probes. The first DNA oligo array 414A will probe for a different DNA sample (e.g., a first single strand of DNA 602) than the second DNA oligo array 414B. In one or more embodiments each well 106 may include a different DNA oligo array 414, allowing for sampling of numerous DNA sequences simultaneously on the same LED panel 101. In one or more embodiments, each well 106 may include the same DNA oligo array 414. In one or more embodiments, the LED panel 101 is integrated into the test device 200 before continuing to operation 504. In one or more embodiments, operation 502 occurs after the LED panel 101 is inserted into the test device.

At operation 504, the sample 610 is prepared for testing. As shown in FIG. 6C, DNA strands have been separated into single strands of DNA (e.g., the first single strand of DNA 602 and the second single strand of DNA 604). DNA may be separated by any reasonable known means (e.g., via helicase enzymes). The single strands of DNA may be a sequence for anything with extractable DNA. For example, the single strand of DNA may be related to a bacterium, virus, or organism. The first single stand of DNA 602 and the second single strand of DNA 604 represent the target DNA. The target DNA is the DNA to be tested by the system described herein. In one or more embodiments, the target DNA may be RNA. Fluorophores (e.g., a first fluorophore 606 and a second fluorophore 608) are bound to the single strands of DNA (e.g., the first single strand of DNA 602 and the second single strand of DNA 604) so that each unique single strand of DNA binds to a fluorophore (e.g., a first fluorophore 606 and a second fluorophore 608). Each fluorophore includes a DNA probe that allows the fluorophore to pair to a single stand of DNA. For example, the first single strand of DNA 602 binds to the first fluorophore 606 via the probe paired to the first fluorophore 606. The first single strand of DNA 602 is labeled with the first fluorophore 606 after the first single strand of DNA 602 binds to the probe of the first fluorophore 606. The first single strand of DNA 602 and the first fluorophore 606 pair together via hydrogen bonds between nucleotides (e.g., C pairs with G and A pairs with T). Each fluorophore (e.g., a first fluorophore 606 and a second fluorophore 608) is excited by UV light 802. For example, a fluorophore that is excited by UV light will emit light. Specifically, for example, Atto425 is excited by UV light and emits light at 485 nm and Atto490LS is excited by UV light 802 and emits light at 661 nm. In certain embodiments the first fluorophore 606 is Atto490LS and the second fluorophore 608 is Atto425. Additionally or alternatively, other fluorophore types that are excited by UV light 802 may be used.

At operation 506, the sample 610 is applied over the LED panel 101. As shown in FIG. 6D, the sample 610 is disposed into each well 106 so that at least the DNA oligo array 414 is submerged in the sample 610. At operation 508, as shown in FIG. 6E, the DNA oligo arrays 414 pair with the single strands of DNA (e.g., the nucleotide 412 sequence of the DNA oligo array 414 pairs with a corresponding sequence of the single strands of DNA (e.g., the first single strand of DNA 602 and the second single strand of DNA 604)). As shown in FIG. 6E, a portion of the sequence (e.g. Seq. ID No. 1 and Seq ID No. 2) are shown for the first single strand of DNA 602 and the second single strand of DNA 604. For example, the second DNA oligo array 414B pairs with the second single strand of DNA 604. Further, the first DNA oligo array 414A pairs with the first single strand of DNA 602. For example, the DNA oligo arrays 414 pair with the single strands of DNA (e.g., the first single strand of DNA 602 and the second single strand of DNA 604) via hydrogen bonds between the nucleotides (e.g., C pairs with G and A pairs with T). The DNA oligo arrays 414 will probe (bind) to only the single strand DNA (e.g., the first single strand of DNA 602 or the second single strand of DNA 604) with a corresponding nucleotide sequence. For example, the second single strand of DNA 604 contains a sequence of CCTTAACC which is an exact binding match for the second DNA oligo array 414B containing GGAATTGG, as shown in well 106A. For example, the first single strand of DNA 602 contains a sequence of GGAATTGG which is an exact bonding match for the first DNA oligo array 414A containing CCTTAACC, as shown in well 106B. Further, in at least this example, only one single strand DNA (e.g., the first single strand of DNA 602 and the second single strand of DNA 604) will bind to the DNA oligo arrays 414.

At operation 510, the sample 610 is removed from the LED panel 101. In one or more embodiments, operation 508 includes a rinse step to remove residual single strand DNA (e.g., the first single strand of DNA 602 and the second single strand of DNA 604) from the LED panel 101. Residual single strands of DNA may include any single strand of DNA (e.g., the first single strand of DNA 602 and the second single strand of DNA 604) that did not pair to a DNA oligo array 414. As shown in FIG. 6F, after operation 508, the first single strand of DNA 602 (including the Seq. ID No. 1) and the second single strand of DNA 604 (including the Seq. ID No. 2) are paired to their respective DNA oligo arrays (e.g., the first DNA oligo array 414A or the second DNA oligo array 414B). At least one fluorophore (e.g. the first fluorophore 606 or the second fluorophore 608) is paired to the respective single strand of DNA (e.g., the first single strand of DNA 602 and the second single strand of DNA 604). Thus, a fluorophore (e.g. the first fluorophore 606 or the second fluorophore 608), a single strand of DNA (e.g., the first single strand of DNA 602 and the second single strand of DNA 604), and a DNA oligo array (e.g., the first DNA oligo array 414A or the second DNA oligo array 414B) are attached to a linker molecule 404 within a well 106 of the LED panel 101. In one or more embodiments, at least one well 106 may be empty (e.g., no sample 610 is disposed in the well 106).

In one or more embodiments, the LED panel 101 including the a fluorophore (e.g. the first fluorophore 606 or the second fluorophore 608), a single strand of DNA (e.g., the first single strand of DNA 602 and the second single strand of DNA 604), and a DNA oligo array (e.g., the first DNA oligo array 414A or the second DNA oligo array 414B) are attached to a linker molecule 404 may be placed under a photo-diode, or any other reasonable means of reading a wavelength of light. For example, the LED panel 101 may be disposed in a test device 200 as described below in method 700. Additionally or alternatively, any other means (e.g., an image sensor) of detecting light emitted from UV light excited fluorophores at varying wavelengths may be used to detect DNA samples disposed within each well.

### Method of Detecting the Target DNA Disposed in a Sample

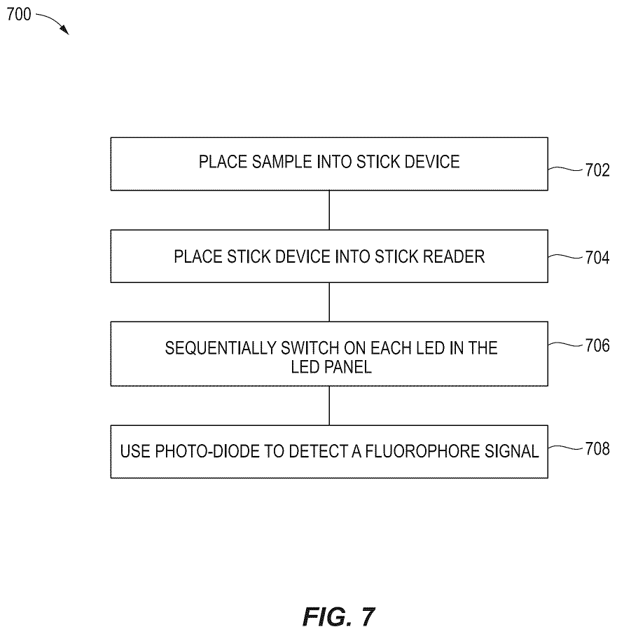

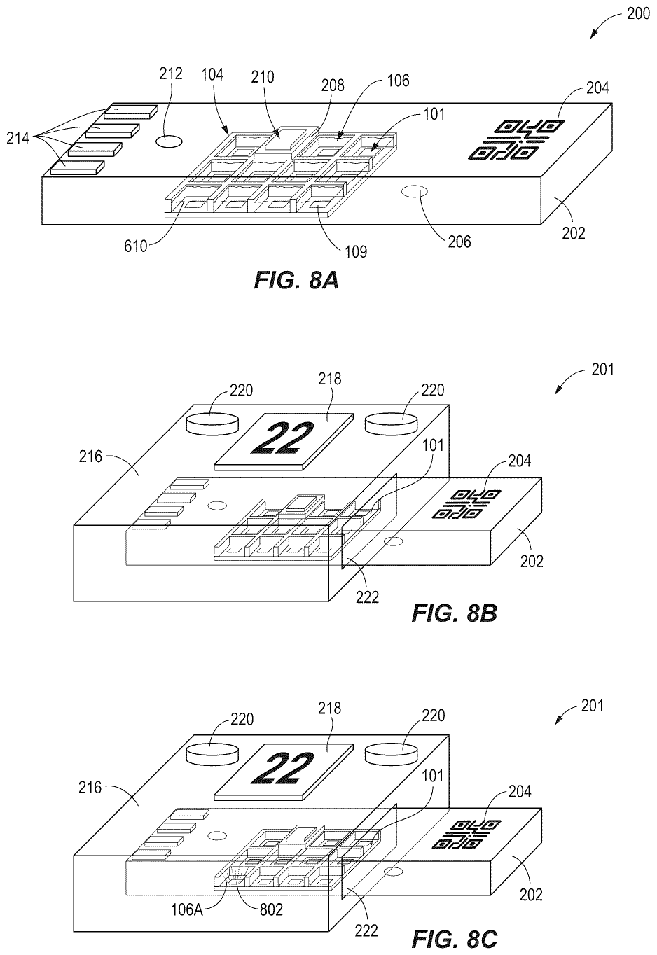

FIG. 7 is a flow diagram of a method 700 of detecting the target DNA disposed in a sample 610. The sample 610 includes at least one single strand DNA (e.g., the first single strand of DNA 602 and/or the second single strand of DNA 604) and at least one fluorophore (e.g. the first fluorophore 606 and/or the second fluorophore 608). The target DNA is, for example, the first single strand of DNA 602 and/or the second single strand of DNA 604. However, it should be understood that any DNA samples may be used. Additionally or alternatively, the sample 610 includes RNA. FIG. 8A-8C is a schematic view of a test device 200 and a test device reader 216 during detection of a sample 610. In one or more embodiments, the test device reader 216 is a computer or smart phone.

At operation 702, the sample 610 is placed into the test device 200. The test device 200 includes a LED panel 101. The LED panel 101 includes DNA oligo arrays 414 prepared according to method 300 as described above in method 300. The sample 610 is prepared according to method 500 as described above. The sample 610 includes at least one single strand of DNA (e.g., the first single strand of DNA 602 and/or the second single strand of DNA 604) and at least one fluorophore (e.g. the first fluorophore 606 and/or the second fluorophore 608). The sample 610 is administered into the test device 200 via a dispensing orifice 212. The sample flows over the LED panel 101 and the sample 610 is disposed over the wells 106 of the LED panel 101. The single strands of DNA (e.g., the first single strand of DNA 602 and the second single strand of DNA 604) pair to a DNA oligo array 414 as described above in method 500. As shown in FIG. 8A, the sample 610 has been administered into the test device 200 and has been disposed over the wells 106 of the LED panel 101. The sample 610 is removed from the LED panel 101 and exits the test device 200 via a drain orifice 206. In certain embodiments, the LED panel 101 is rinsed. Rinsing fluid will enter the test device 200 via the dispensing orifice 212 and exit the test device 200 via the drain orifice 206. After the rinse step, single strands of DNA (e.g., the first single strand of DNA 602 and the second single strand of DNA 604) and fluorophores (e.g. the first fluorophore 606 or the second fluorophore 608) remain in each well paired to DNA oligo arrays 414 and bound to the glass surface of the LEDs 109. The single strands of DNA may be a sequence for anything with extractable DNA. For example, the single strand of DNA may be related to a bacterium, a virus or an organism.

At operation 704, as shown in FIG. 8B, the test device 200 is placed into the test device reader 216 to form the DNA sampling system 201. The plurality of contact pins 214 allow for an electrical connection to form between the test device 200 and the test device reader 216. The display 218 may indicate what type of test device 200 has been inserted into the test device reader 216. The photo-diode 210 is positioned in the test device reader 216 to provide information to the test device reader 216. A user can turn on the system by switching on the test device reader 216 with the switch 220. The switch 220 may also control the plurality of LEDs disposed in the LED panel 101. In one or more embodiments, an application on a user device may control the test device reader 216. In one or more embodiments, the test device reader 216 may couple to a user device. In one or more embodiments, the test device reader 216 is a smartphone, a tablet, or a computer.

At operation 706, as shown in FIG. 8C, each LED 109 is sequentially switched on in the LED panel 101 in order emit UV light 802. For example, as shown in FIG. 8C, UV light 802 is emitted from the LED 109 disposed in well 106A in the LED panel 101. The UV light 802 emits upward toward the target, which is disposed in the well 106. The UV light 802 excites the fluorophore (e.g. the first fluorophore 606 or the second fluorophore 608) paired to the target. The target is the combination of the DNA oligo arrays formed (e.g., the first DNA oligo array 414A or the second DNA oligo array 414B), the single strands of DNA (e.g., the first single strand of DNA 602 and/or the second single strand of DNA 604), and the fluorophore labeled probes (e.g. the first fluorophore 606 or the second fluorophore 608), as described above. The fluorophore (e.g. the first fluorophore 606 or the second fluorophore 608) emits light at a certain wavelength. At operation 708, a photo-diode 210 detects the light emitted from the fluorophore (e.g. the first fluorophore 606 or the second fluorophore 608). A UV filter 208 is disposed over the LED panel 101. The UV filter 208 reflects UV light 802 away from the photo-diode 210 to prevent signal errors of light emission. Additionally or alternatively, the UV filter 208 absorbs the UV light 802. The photo-diode 210 is operable to detect light emitted from a plurality of wells 106. Further, the PI structures 104 protect adjacent wells 106 from light cross talk. This allows the photo-diode 210 to read a signal from each well 106 individually. The photo-diode 210 includes the appropriate connections to convert the received signal to a result, allowing for the test device reader 216 to display the result of on the display 218. In one or more embodiments, the test device reader is a computer or smart phone capable of pairing through a USB port or USB-C port. In one or more embodiments, a software application is operable to pair with the test device.

For example, the LED panel 101 disposed within the test device 200 includes a well 106A. A sample 610 (prepared according to method 500) is disposed in the well 106A. As an example, the sample 610 includes DNA from the salmonella bacteria. However, in other embodiments the sample 610 can include any type of DNA. The sample 610 includes the second single strand of DNA 602 and the second fluorophore 608. In this example, the second single strand of DNA 602 is DNA from the salmonella bacteria. The sample 610 is administered into the LED panel 101 via the dispensing orifice 212 of the test device 200. The sample 610 is paired to the first DNA oligo array 414A. The first DNA oligo array 414A, which is paired to the linker molecule 404, pairs to the second single strand of DNA 602, therefore, bind the second single strand of DNA 602 in the well 106A. Residual sample 610 is removed from LED panel and the test device 200. An optional rinse step may be performed. The user inserts the test device 200 into the test device reader 216 and prompts the test device reader 216 to begin testing (e.g., switching the device on via the switch 220). The LED panel 101 individually switches each LED 109 on sequentially. Each LED 109 emits a UV light 802. For example, the LED 109 disposed in well 106A emits a UV light 802. The UV light 802 excites the second fluorophore 608. The excited second fluorophore 608 emits a light at a certain wavelength (e.g., Atto490LS is excited by UV light and emits light at 661 nm) that is read by the photo-diode 210. The photo-diode 210 provides the signal (e.g., light detection) to the test device reader 216. The test device reader 216 interprets the signal (e.g., light detection at a certain wavelength) and translates it to a result that indicates if a DNA sequence was detected in well 106A. In this example, the test device reader 216 displays the result to the display 218 to indicate to the user that salmonella DNA (e.g., the second single strand of DNA 602) was detected in the sample 610 disposed in well 106A.

It is contemplated that each well 106 can include a different single strand of DNA. Therefore, allowing for many types of samples to be tested at once. In one or more embodiments, two or more photo-diodes 210 may be disposed over the LED panel 101 of the test device 200 in order to decrease processing time and result reporting time. In an embodiment including two or more photo-diodes 210 and an additional light filter is used to filter out light emitted at different wavelengths from different fluorophores.

Overall, embodiments of the present invention generally relate to devices and methods related to DNA oligo manufacturing and DNA sample detection. Specifically, embodiments disclosed herein provide devices and methods for manufacturing DNA and a detection device associated with DNA sequence detection that incorporate a light emitting diode (LED) panel. The LED panel allows for a simple method and device for controlled growth of each DNA oligo array without the use of photolithography tools such as masks. Further, administering samples over the LED panel and using the same LED panel, not only for DNA oligo array manufacturing, but for sample testing allows for easy, cost effective detection of particular DNA sequences while minimizing the equipment needed to produce a result.

While the foregoing is directed to embodiments of the present disclosure, other and further embodiments of the disclosure may be devised without departing from the basic scope thereof, and the scope thereof is determined by the claims that follow.

What is claimed is:

1. A device for synthesizing deoxyribonucleic acid (DNA) comprising:
a LED panel, the LED panel comprising:
a backplane, the backplane having a backplane surface, the backplane surface having at least one contact pad disposed over the backplane surface;
a plurality of LEDs, the plurality of LEDs disposed over the backplane surface, wherein each LED of the plurality of LEDs couples to a contact pad; and
pixel isolation (PI) structures, the PI structures disposed over the backplane surface and between the plurality of LEDs, the PI structures defining a plurality of wells; and
a coating disposed over the LED panel; the coating operable to seal each well to hold a liquid.

2. The device of claim 1, wherein further comprising a cover disposed over the LED panel and in each well of the plurality of wells.

3. The device of claim 1, wherein the plurality of LEDs emit UV light.

4. The device of claim 1, wherein the coating is a SiO₂ containing film.

5. The device of claim 1, wherein the PI structures comprise an organic material, the organic material operable to filter or absorb light.

6. The device of claim 1, further comprising:
a dispensing orifice;
a drain orifice;
at least one light filter;
at least one photosensitive device; and
a plurality of contact pins.

7. The device of claim 6, wherein the plurality of contact pins provide means for an electrical connection to a test device reader.

8. A device comprising:
a LED panel, the LED panel comprising:
a backplane, the backplane having a backplane surface, the backplane surface having at least one contact pad disposed over the backplane surface;
a plurality of LEDs, the plurality of LEDs disposed over the backplane surface, wherein each LED of the plurality of LEDs couples to a contact pad; and
pixel isolation (PI) structures, the PI structures disposed over the backplane surface and between the plurality of LEDs, the PI structures defining a plurality of wells, wherein each LED of the plurality of LEDs disposed in each well of the plurality of wells is operable to anchor deoxyribonucleic acid (DNA) oligo array for DNA synthesis.

9. A device comprising:
a LED panel, the LED panel comprising:
a backplane, the backplane having a backplane surface, the backplane surface having at least one contact pad disposed over the backplane surface;
a plurality of LEDs, the plurality of LEDs disposed over the backplane surface, wherein each LED of the plurality of LEDs couples to a contact pad; and
pixel isolation (PI) structures, the PI structures disposed over the backplane surface and between the plurality of LEDs, the PI structures defining a plurality of wells;
a coating disposed over the LED panel; the coating operable to seal each well to hold a liquid; and
deoxyribonucleic acid (DNA) directly synthesized on each LED of the plurality of LEDs or synthesized over the coating disposed over the LED panel.

10. A method for fabricating deoxyribonucleic acid (DNA), the method comprising:
applying a starting medium over a LED panel;
applying a plurality of linker molecules over the LED panel, each linker molecule of the plurality of linked molecules attaching over a LED;
applying a plurality of photosensitive molecules over the LED panel, the plurality of photosensitive molecules being sensitive to a UV light, at least one photosensitive molecule of the plurality of photosensitive molecules binding to the plurality of linker molecules;
cleaving the at least one photosensitive molecule of the plurality of photosensitive molecules from at least one linker molecule of the plurality of linker molecules with the UV light;
applying a nucleotide solution over the LED panel, a nucleotide from the nucleotide solution attaching to the at least one linker molecule of the plurality of linker molecules;
rinsing the nucleotide solution from the LED panel; and
applying the plurality of photosensitive molecules over the LED panel, at least one photosensitive molecule of the plurality of photosensitive molecules binding to the nucleotide.

11. The method of claim 10, wherein the LED panel comprises:
a backplane, the backplane having a backplane surface, the backplane surface having at least one contact pad disposed over the backplane surface;
a plurality of LEDs, the plurality of LEDs disposed over the backplane surface, wherein each LED of the plurality of LEDs couples to a contact pad;
pixel isolation (PI) structures, the PI structures disposed over the backplane surface and between the plurality of LEDs, the PI structures defining a plurality of wells; and
a coating disposed over the LED panel, the coating operable to seal each well to hold a liquid.

12. The method of claim 11, wherein the coating is a SiO₂ containing film.

13. The method of claim 11, further comprising:
cleaving the at least one photosensitive molecule of the plurality of photosensitive molecules from at least one linker molecule of the plurality of linker molecules with the UV light;
applying the nucleotide solution over the LED panel, a nucleotide from the nucleotide solution attaching to the at least one linker molecule of the plurality of linker molecules;
rinsing the nucleotide solution from the LED panel;
applying the plurality of photosensitive molecules over the LED panel, at least one photosensitive molecule of the plurality of photosensitive molecules binding to the nucleotide; and
repeating the method to form a DNA oligonucleotide.

14. The method of claim 13, further comprising:
applying the nucleotide solution comprising a plurality of nucleotides over the LED panel, the plurality of nucleotides pairing with complementary nucleotides of the DNA oligonucleotide; and
forming DNA.

15. The method of claim 14, further comprising:
cleaving the DNA from each LED of the plurality of LEDs disposed in each well of the plurality of wells.

16. The method of claim 14, wherein the nucleotide solution includes a plurality of a same nucleotide or a plurality of different nucleotides.

17. The method of claim 10, wherein the starting medium comprises silane.

18. The method of claim 10, wherein the UV light is emitted from each LED of a plurality of LEDs.

19. The method of claim 11, wherein each LED of the plurality of LEDs includes a DNA oligo array, DNA, or combinations thereof that is a same sequence, a different sequence, or combinations thereof.

20. The method of claim 14, wherein forming DNA comprises forming a phosphodiester bond between the DNA oligonucleotide and a plurality of complementary nucleotides from the nucleotide solution.


SEQUENCE LISTING (2 sequences)

SEQ ID NO: 1 - DNA, 40 bp, synthetic construct
  source 1..40: mol_type = other DNA
atgacggatcagccgcaagcggaattggcgacataacaag

SEQ ID NO: 2 - DNA, 40 bp, synthetic construct
  source 1..40: mol_type = other DNA
tactgcctagtcggcgttcgccttaaccgctgtattgttc